(19) United States
(12) Patent Application Publication
(10) Pub. No.: US 2023/0001154 A1
(43) Pub. Date: Jan. 5, 2023

(54) TUBING ANCHOR

(71) Applicant: CAREFUSION 303, INC., San Diego, CA (US)

(72) Inventors: Jason Andrew WINE, Brea, CA (US); Siddarth K. SHEVGOOR, Mission Viejo, CA (US)

(21) Appl. No.: 17/853,815

(22) Filed: Jun. 29, 2022

Related U.S. Application Data
(60) Provisional application No. 63/217,160, filed on Jun. 30, 2021.

(51) Int. Cl. A61M 25/02

(52) U.S. Cl. CPC A61M 25/02; A61M 2025/028; A61M 2025/0266

(57) ABSTRACT

Tubing anchors are disclosed that can include an anchor body configured to couple with a length of tubing and to be coupled with a patient to resist unintended separation of the tubing from the patient, where the tubing anchor can include any of a sidewall, a top surface, and a bottom surface configured to define a surface area to engage against any of a tape and adhesive to resist unintended separation of the anchor body from the patient.

### CROSS-REFERENCE TO RELATED APPLICATIONS

This application claims the benefit of U.S. Provisional Application No. 63/217,160, filed Jun. 30, 2021, the entirety of which is incorporated herein by reference.

### BACKGROUND

The present disclosure relates generally to devices for accessing an internal portion of a patient through tubing, and, more particularly, to tubing anchors that can be coupled with tubing and affixed to a patient to resist unintended separation of the tubing or the device for accessing the patient.

Tubing can be used for a variety of therapies that may require accessing an internal portion of a patient, including, for example intravenous therapy and enteral feeding. An internal portion of a patient can be accessed by creating a pathway that penetrates or enters into the patient through a natural or artificial opening of the patient. The pathway can be created or maintained using a device, such as a catheter coupled to tubing, or tubing itself. Often, an extension set is coupled to a catheter to provide one or more access point into the pathway coupled to the patient.

In some instances, the device, tubing, or extension set can be unintentionally separated when an unintended or unexpected force is exerted on the device. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

To resist or prevent unintended separation of the device, tubing, or extension set from the patient, any of the medical device and the tubing can be affixed to the patient's skin. Tape or an adhesive can be implemented to affix the device or tubing to the patient's skin; however, such methods have limited effectiveness. The tape utilized for affixing the device or tubing to the patient is often intended to be cut or torn by hand instead of requiring scissors; however, such characteristics provide a tape with a low shear strength. As such, when the tubing is pulled, relative away from the patient, the tape can tear and thereby reduce the resistance to separation of the tubing from the patient.

Further, the tubing used for access into a patient, including the tubing used for extension sets, can have a narrow diameter or width. As such, the surface area engaged by the tape against the tubing is small, relative to the surface area necessary to resist separation from the patient. As a result, the tubing can tear or shear through the tape, or the tubing can move or be pulled from under the tape.

In accordance with at least some embodiments disclosed herein is the realization that unintended dislodgement or disconnection of a medical connection, such as a medical tubing, can result in injury to a patient or a caregiver, such as by depriving the patient of a medicament, increasing the potential for infection to the patient, and exposing the caregiver to harmful medicaments.

Accordingly, an aspect of the present disclosure provides a tubing anchor comprising an anchor body having a first end, a second end, a first sidewall, a second sidewall, a top end, a bottom end, and a tubing passage extending through the first and second ends and defining a longitudinal axis of the anchor body, the first sidewall having front and rear portions that extend away from the longitudinal axis and intersect at an apex of the first sidewall, the second sidewall having front and rear portions that extend away from the longitudinal axis and intersect at an apex of the second sidewall, wherein a width of the anchor body between the apex of the first and second sidewalls, is greater than a height of the anchor body between the top and bottom ends.

Some instances of the present disclosure provide a tubing anchor comprising an anchor body having a first end, a second end, a top end, a bottom end, a first wing, a second wing, and a tubing passage extending through the first and second ends and defining a longitudinal axis of the anchor body, each of the first and second wings comprising a proximal portion adjacent to the longitudinal axis and an apex that is distal to the longitudinal axis, wherein a width of the anchor body defined between the distal end portions of the first and second wings is greater than a height of the anchor body between the top and bottom ends.

In some aspects, the present disclosure provide intravenous tubing anchor system comprising: a tubing having a longitudinal length; and an anchor body having a first end, a second end, a first sidewall, a second sidewall, a top end, a bottom end, and a tubing passage extending through the first and second ends and defining a longitudinal axis of the anchor body, the first sidewall having a front and rear portions that extend away from the longitudinal axis and intersect at an apex of the first sidewall, the second sidewall having a front and rear portions that extend away from the longitudinal axis and intersect at an apex of the second sidewall, wherein a width of the anchor body between the apex of the first and second sidewalls, is greater than a height of the anchor body between the top and bottom ends; wherein the tubing is positioned within the tubing passage such that a portion of the longitudinal length of the tubing is coextensive with the longitudinal axis of the anchor body

Accordingly, the present application addresses several operational challenges encountered in prior systems and methods for coupling medical tubing to a patient and provides numerous improvements that enable a user to more securely and easily couple a tubing to patient while increasing resisting to unintended dislodgement or disconnection of the tubing.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a tubing anchor in use with an IV set coupled to a patient, in accordance with aspects of the present disclosure.

FIG. 2 illustrates a tubing anchor coupled to an IV extension set, in accordance with aspects of the present disclosure.

FIG. 3 illustrates a front perspective view of a tubing anchor, in accordance with aspects of the present disclosure.

FIG. 4 illustrates a top elevation view of a tubing anchor, in accordance with aspects of the present disclosure.

FIGS. 5 and 6 illustrate rear perspective views of a tubing anchor, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a needleless fluid connector system. The needleless fluid connector system can provide for efficient and safe maintenance of fluid connections, such as the connections used for transferring medical fluids toward or away from a patient. The needleless fluid connector system can maintain a fluid pathway by resisting unintended disconnection when a pulling or tension force is applied to the needleless fluid connector system, such as when a patient moves or when the medical tubing is pulled away from the patient.

The needleless fluid connector system can also prevent injury to a patient or a caregiver by permitting disconnection or separation between portions of the connector system when a pulling or tension force exceeds a threshold. The needleless fluid connector system can also prevent injury to a patient or a caregiver by obstructing the fluid pathway when disconnection or separation between portions of the connector system occurs. Further, the needleless fluid connector system can provide for efficient and safe reestablishment of the fluid pathway, by permitting reassembly of portions of the system after a disconnection or separation occurs.

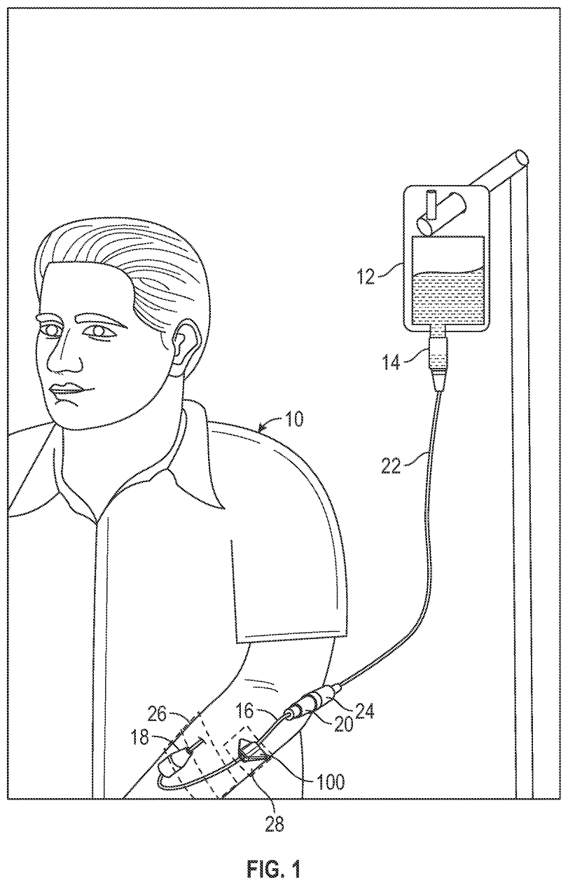

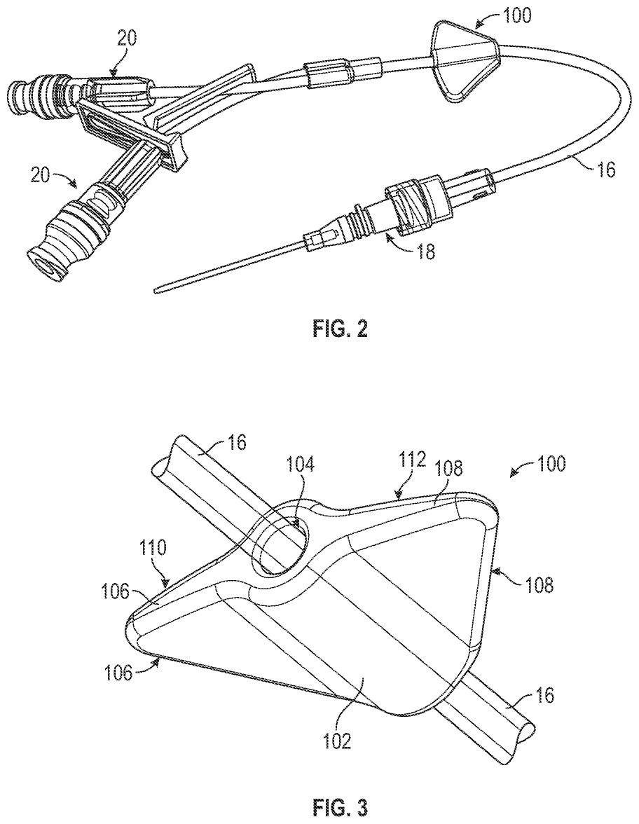

Referring now to the figures, FIGS. 1 and 2 illustrate an example of a tubing anchor 100 in use in accordance with aspects of the present disclosure. The tubing anchor 100 is coupled with an IV extension set which includes tubing 16 and a catheter 18. A portion of the catheter 18 is inserted into the patient and a connector 20 of the IV extension set is coupled to tubing 22 of an IV set.

The IV extension set is fluidly coupled with an IV set that includes a medicament bag 12, a drip chamber 14, tubing 22, and a connector 24. The connector 24 of the IV set is fluidly coupled to the connector 20 of the IV extension set. The IV set and IV extension set are configured to direct a fluid toward or away from the patient 10.

To resist unintended dislodgement or disconnection of the tubing 16 or the catheter 18 from the patient, tape 26 is placed over the tubing 16 and the catheter 18, so that the tape engages the tubing 16, the catheter 18, and the patient. To increase the resistance to unintended dislodgement or disconnection of the tubing 16 or catheter 18, a tubing anchor 100 is positioned along the tubing 16, and tape 28 is placed over the tubing anchor 100 and the tubing 16, so that the tape engages the tubing 16, the tubing anchor 100, and the patient.

In use, if the tubing 16, 22, or another portion IV extension and IV set between the tubing anchor 100 and the medicament bag 12, is pulled in a direction away from the tubing anchor, the tape 28, which is engaged against the tubing anchor 100 and the patient 10, can resist the force and prevent unintended dislodgement or disconnection of tubing anchor 100 and the catheter 18 from the patient.

To resist unintended dislodgement or disconnection, the tubing anchor 100 includes an anchor body with an inner surface forming a tubing passage and an outer surface forming a sidewall, where the sidewall extends in a direction away from the tubing passage. The outer surface of the anchor body can provide a larger surface area, relative to the surface area of the tubing along a coextensive length of the tubing anchor, for engagement by the tape. In some embodiments of the present disclosure, the tubing anchor 100 can include first and second sidewalls that extend in a direction away from the tubing passage.

Although the tubing anchor 100 is illustrated as being coupled along a fluid pathway of an IV extension set, it should be understood that the tubing anchor 100 can be connected to other fluid pathways, such as IV tubing, an IV set, or an enteral feeding tube.

An embodiment of the tubing anchor 100 is illustrated in FIG. 3. The tubing anchor 100 includes an anchor body 102 with an inner surface that forms a tubing passage 104 extending through the anchor body 102. The tubing passage 104 can have a cross-sectional diameter that is approximately equal to or greater than a diameter defined by the outer surface of the tubing 16.

The anchor body 102 also includes a first sidewall 106 and a second sidewall 108, where each of the first and second sidewalls 106, 108 extend in a direction away from the tubing passage 104. In some embodiments of the present disclosure, the first and second sidewalls 106, 108 are shaped to form first and second wings 110, 112, respectively.

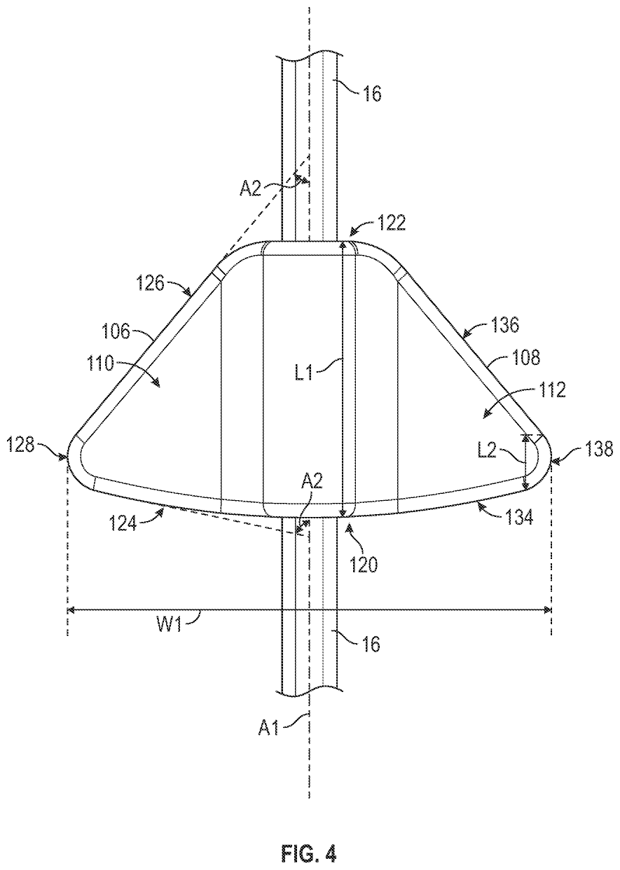

Referring to the top elevation of view of the tubing anchor 100 in FIG. 4, the tubing passage 104 extends through a first end 120 and a second end 122 of the anchor body and defines a longitudinal axis A1 through the anchor body. The first and second sidewalls 106, 108 extend in a direction away from the longitudinal axis A1. The first sidewall 106 extends in a first direction away from the longitudinal axis A1, and the second sidewall 108 extends in a second direction away from the longitudinal axis A1, where the second direction is different than the first direction.

The first sidewall 106 also extends between the first end 120 and the second end 122 of the anchor body, and the second sidewall 108 extends between the first end 120 and the second 122 of the anchor body. The first sidewall 106 includes a front portion 124 and a rear portion 126, where the front portion 124 extends away from the first end 120, and the rear portion 126 extends away from the second end 122. The front and rear portions 124, 126 intersect at an apex 128 of the first sidewall.

The second sidewall 108 includes a front portion 134 and a rear portion 136, where the front portion 134 extends away from the first end 120, and the rear portion 136 extends away from the second end 122. The front and rear portions 134, 136 intersect at an apex 138 of the second sidewall.

The front portions 124, 134 and the rear portions 126, 136 of the first and second sidewalls 106, 108, respectively, can extend toward each other to form a wing shape that tapers away from the longitudinal axis A1. The wings 110, 112 can be configured with a proximal portion adjacent to the longitudinal axis A1 and an apex that is distal to the longitudinal axis A1. In embodiments in which the wing shape that tapers away from the longitudinal axis A1, the anchor body can have a first length L1, adjacent the tubing passage, that decreases in a direction away from the longitudinal axis A1 toward a second length L2.

In some aspects of the present disclosure, the front portions 124, 134 of the first and second sidewalls 106, 108, respectively, extends away from the longitudinal axis A1 at a first angle A2 therebetween, and the rear portions 126, 136 of the first and second sidewalls 106, 108, respectively, extends away from the longitudinal axis A1 at a second angle A3 therebetween. The first and second angles A2, A3 can be selected such that the front and rear portions of the sidewalls 106, 108 intersect. In some embodiments, the first angle A2 is greater than the second angle A3.

Although the tubing anchor 100 is illustrated having an anchor body 102 with first and second sidewalls 106, 108 forming first and second wings, it should be understood that in some embodiments of the present disclosure an anchor body 102 having one sidewall or more than two sidewalls is contemplated. In some embodiments of the present disclosure, it is also contemplated that the wings can be configured to have a length that increases in a direction away from the longitudinal axis A1.

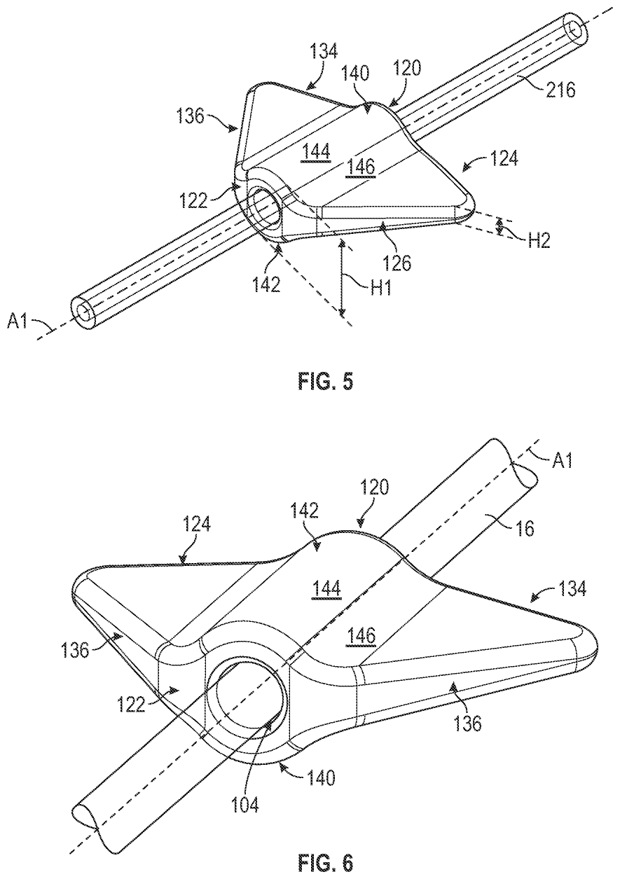

The anchor body 102 further defines a top end 140 and a bottom end 142 of the tubing anchor, as illustrated in FIGS. 5 and 6. The top end 140, illustrated in FIG. 5, can include at least a portion a top surface of the tubing anchor 100, and the bottom end 142, illustrated in FIG. 6, can include at least a portion a bottom surface of the tubing anchor 100.

Any of the top and bottom surfaces of the tubing anchor 100 can form any of a convex and/or concave shape. The top surface can form a convex shape 144 that extends along the tubing passage, between the first and second ends 120, 122 of the anchor body. A portion of the top surface can also form a concave shape 146 that is adjacent to the tubing passage. The convex shape 144 can extend between the first and second ends 120, 122 of the anchor body, and be positioned between portions of the outer surface that have a concave shape 146.

The bottom surface can form a profile shape with convex and concave shapes that mirror the top surface. In some embodiments of the present disclosure, the top and bottom surfaces can have the same or different profile shapes. For example, the top surface can include a convex shape and the bottom surface can form a concave shape, where the convex shape of the top surface can provide for an anchor body with a low profile to avoid being bumped or pulled unintentionally while providing a large surface area relative to the height of the anchor body. The concave shape of the bottom surface that corresponds with curved surface defined by a patient's body thereby increasing patient comfort.

The top and bottom ends 140, 142 of the anchor body 102 define a height H1 of the anchor body therebetween, and the apex 128, 138 of the first and second sidewalls define a width W1 of the anchor body therebetween. The width W1 can be greater than the height H1 of the anchor body, where the width W1 and length L1 can be configured to provide a larger surface are for engaging the tubing anchor against a length of tape and against the patient.

In some embodiments, the height of the anchor body decreases in a direction away from the longitudinal axis A1. For example, a concave portion of the outer surface of the anchor body can form a portion the height H1 that tapers away from the longitudinal axis A1. In some embodiments, the width of the anchor body decreases between the first and second ends 120, 122. For example, the width of the anchor body can decrease in a direction from the first end 120 toward the second end 122, thereby providing a wider cross-sectional profile of the first end 120, relative to the second end 122. The wider cross-sectional profile of the first end 120 can provide resistance to unintentional separation of the tubing from the patient when the tubing anchor 100 is pulled in a direction from the second end 122 toward the first end 120.

The anchor body 102, or portion thereof, can be formed from a soft plastic, such as a thermoplastic elastomer (TPE). In some embodiments, a portion of the anchor body 102 can be more flexible, relative to another portion thereof. For example, a portion of the anchor body 102 proximal to an apex of a sidewall can have a height H2 that is less than the H1, such that the portion of the anchor body 102 proximal to an apex of a sidewall is more flexible relative to the portion of the anchor body 102 having the height H1. The flexible portion of the anchor body 102 can be configured to be biased or flexed relative to the skin of a patient to more closely correspond to the patient's body.

In use, the tubing anchor 100 can be coupled to a length of tubing by inserting the tubing through the tubing passage 104. The tubing anchor 100 can then be moved or slid along the tubing 16 to a desired position. The tubing anchor 100 can then be affixed to the patient by taping the tubing anchor 100 to the patient or by exposing an adhesive on an outer surface of the tubing anchor.

In some embodiments of the present disclosure, an end of a length of tubing 16 can be coupled to the tubing passage 104. In some embodiments, an end of a first length of tubing and an end of a second length of tubing can be coupled to openings of the tubing passage 104 at the first and second ends 120, 122 of the anchor body, respectively. In some aspects of the present disclosure, first and second ends of the tubing are spaced apart along the tubing passage 104, and in some embodiments of the present disclosure, the first and second ends of the tubing are engaged against each other within the tubing passage 104. In some embodiments of the present disclosure, the inner surface of the anchor body 102 along the tubing passage 104 forms a portion of a fluid passageway through the anchor body.

When another portion of the tubing spaced apart from the tubing anchor 100, or another medical device coupled to the tubing, is pulled, engagement of the tape or adhesive against the tubing anchor 100 can resist separation of the tubing from the patient. In some instances, the tubing anchor 100 can permit another portion of the IV extension set, such as the connector 20 to deflect or move relative to the tubing anchor 100.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A tubing anchor comprising an anchor body having a first end, a second end, a first sidewall, a second sidewall, a top end, a bottom end, and a tubing passage extending through the first and second ends and defining a longitudinal axis of the anchor body, the first sidewall having front and rear portions that extend away from the longitudinal axis and intersect at an apex of the first sidewall, the second sidewall having front and rear portions that extend away from the longitudinal axis and intersect at an apex of the second sidewall, wherein a width of the anchor body between the apex of the first and second sidewalls, is greater than a height of the anchor body between the top and bottom ends.

Clause 2. The tubing anchor of Clause 1, wherein a length of the anchor body between the first and second ends and along the longitudinal axis is greater than the height of the anchor body.

Clause 3. The tubing anchor of Clause 1, wherein the front and rear portions of the first sidewall extend toward each other such that a length of the anchor body decreases along the first sidewall in a direction away from the longitudinal axis.

Clause 4. The tubing anchor of Clause 1, wherein the front portion of the first sidewall extends away from the longitudinal axis at a first angle therebetween, and the rear portion of the first sidewall extends away from the longitudinal axis at a second angle therebetween, and wherein the first angle is greater than the second angle.

Clause 5. The tubing anchor of Clause 1, wherein the height of the anchor body decreases in a direction away from the longitudinal axis.

Clause 6. The tubing anchor of Clause 1, wherein the first sidewall extends in a first direction away from the longitudinal axis, and wherein the second sidewall extends in a second direction away from the longitudinal axis, wherein the second direction is different than the first direction.

Clause 7. The tubing anchor of Clause 1, wherein an outer surface of the anchor body along any of the top and bottom ends define a convex surface along the longitudinal axis.

Clause 8. The tubing anchor of Clause 1, wherein an outer surface of the anchor body along any of the top and bottom ends define a concave surface adjacent to the longitudinal axis.

Clause 9. The tubing anchor of Clause 1, wherein a length of the anchor body defined between the front and rear portions of the first sidewall tapers in a direction away from the longitudinal axis.

Clause 10. A tubing anchor comprising an anchor body having a first end, a second end, a top end, a bottom end, a first wing, a second wing, and a tubing passage extending through the first and second ends and defining a longitudinal axis of the anchor body, each of the first and second wings comprising a proximal portion adjacent to the longitudinal axis and an apex that is distal to the longitudinal axis, wherein a width of the anchor body defined between the distal end portions of the first and second wings is greater than a height of the anchor body between the top and bottom ends.

Clause 11. The tubing anchor of Clause 10, wherein a length of the anchor body between the first and second ends and along the longitudinal axis is greater than the height of the anchor body.

Clause 12. The tubing anchor of Clause 10, wherein the height of the anchor body decreases in a direction away from the longitudinal axis.

Clause 13. The tubing anchor of Clause 10, wherein an outer surface of the anchor body along any of the top and bottom ends define a convex surface along the longitudinal axis.

Clause 14. The tubing anchor of Clause 10, wherein an outer surface of the anchor body along any of the top and bottom ends define a concave surface adjacent to the longitudinal axis.

Clause 15. The tubing anchor of Clause 10, wherein a length of the anchor body between the first and second ends tapers in a direction away from the longitudinal axis.

Clause 16. The tubing anchor of Clause 10, wherein an outer surface of the anchor body comprises an adhesive.

Clause 17. An intravenous tubing anchor system comprising: a tubing having a longitudinal length; and an anchor body having a first end, a second end, a first sidewall, a second sidewall, a top end, a bottom end, and a tubing passage extending through the first and second ends and defining a longitudinal axis of the anchor body, the first sidewall having a front and rear portions that extend away from the longitudinal axis and intersect at an apex of the first sidewall, the second sidewall having a front and rear portions that extend away from the longitudinal axis and intersect at an apex of the second sidewall, wherein a width of the anchor body between the apex of the first and second sidewalls, is greater than a height of the anchor body between the top and bottom ends; wherein the tubing is positioned within the tubing passage such that a portion of the longitudinal length of the tubing is coextensive with the longitudinal axis of the anchor body

Clause 18. The tubing anchor of Clause 17, wherein a portion of the first and second sidewalls, distal to longitudinal axis, is more flexible, relative to another portion of the anchor body proximal to the longitudinal axis.

Clause 19. The tubing anchor of Clause 17, wherein the tubing passage is formed by an inner surface of the anchor body having a cross-sectional width that is transverse relative to the longitudinal axis, and wherein the cross-sectional width of the tubing passage is approximately equal to or less that a cross-sectional width of an outer surface of the tubing.

Clause 20. The tubing anchor of Clause 17, wherein the tubing and the anchor body are bonded together to resist movement of the tubing relative to the anchor body.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A tubing anchor comprising an anchor body having a first end, a second end, a first sidewall, a second sidewall, a top end, a bottom end, and a tubing passage extending through the first and second ends and defining a longitudinal axis of the anchor body,
the first sidewall having front and rear portions that extend away from the longitudinal axis and intersect at an apex of the first sidewall,
the second sidewall having front and rear portions that extend away from the longitudinal axis and intersect at an apex of the second sidewall,
wherein a width of the anchor body between the apex of the first and second sidewalls, is greater than a height of the anchor body between the top and bottom ends.

2. The tubing anchor of claim 1, wherein a length of the anchor body between the first and second ends and along the longitudinal axis is greater than the height of the anchor body.

3. The tubing anchor of claim 1, wherein the front and rear portions of the first sidewall extend toward each other such that a length of the anchor body decreases along the first sidewall in a direction away from the longitudinal axis.

4. The tubing anchor of claim 1, wherein the front portion of the first sidewall extends away from the longitudinal axis at a first angle therebetween, and the rear portion of the first sidewall extends away from the longitudinal axis at a second angle therebetween, and wherein the first angle is greater than the second angle.

5. The tubing anchor of claim 1, wherein the height of the anchor body decreases in a direction away from the longitudinal axis.

6. The tubing anchor of claim 1, wherein the first sidewall extends in a first direction away from the longitudinal axis, and wherein the second sidewall extends in a second direction away from the longitudinal axis, wherein the second direction is different than the first direction.

7. The tubing anchor of claim 1, wherein an outer surface of the anchor body along any of the top and bottom ends define a convex surface along the longitudinal axis.

8. The tubing anchor of claim 1, wherein an outer surface of the anchor body along any of the top and bottom ends define a concave surface adjacent to the longitudinal axis.

9. The tubing anchor of claim 1, wherein a length of the anchor body defined between the front and rear portions of the first sidewall tapers in a direction away from the longitudinal axis.

10. A tubing anchor comprising an anchor body having a first end, a second end, a top end, a bottom end, a first wing, a second wing, and a tubing passage extending through the first and second ends and defining a longitudinal axis of the anchor body, each of the first and second wings comprising a proximal portion adjacent to the longitudinal axis and an apex that is distal to the longitudinal axis, wherein a width of the anchor body defined between the apex of the first and second wings is greater than a height of the anchor body between the top and bottom ends.

11. The tubing anchor of claim 10, wherein a length of the anchor body between the first and second ends and along the longitudinal axis is greater than the height of the anchor body.

12. The tubing anchor of claim 10, wherein the height of the anchor body decreases in a direction away from the longitudinal axis.

13. The tubing anchor of claim 10, wherein an outer surface of the anchor body along any of the top and bottom ends define a convex surface along the longitudinal axis.

14. The tubing anchor of claim 10, wherein an outer surface of the anchor body along any of the top and bottom ends define a concave surface adjacent to the longitudinal axis.

15. The tubing anchor of claim 10, wherein a length of the anchor body between the first and second ends tapers in a direction away from the longitudinal axis.

16. The tubing anchor of claim 10, wherein an outer surface of the anchor body comprises an adhesive.

17. An intravenous tubing anchor system comprising:
a tubing having a longitudinal length; and
an anchor body having a first end, a second end, a first sidewall, a second sidewall, a top end, a bottom end, and a tubing passage extending through the first and second ends and defining a longitudinal axis of the anchor body, the first sidewall having a front and rear portions that extend away from the longitudinal axis and intersect at an apex of the first sidewall, the second sidewall having a front and rear portions that extend away from the longitudinal axis and intersect at an apex of the second sidewall, wherein a width of the anchor body between the apex of the first and second sidewalls, is greater than a height of the anchor body between the top and bottom ends;
wherein, at least a portion of the longitudinal length of the tubing is coupled with the tubing passage.

18. The tubing anchor of claim 17, wherein a portion of the first and second sidewalls, distal to longitudinal axis, is more flexible, relative to another portion of the anchor body proximal to the longitudinal axis.

19. The tubing anchor of claim 17, wherein the tubing passage is formed by an inner surface of the anchor body having a cross-sectional width that is transverse relative to the longitudinal axis, and wherein the cross-sectional width of the tubing passage is approximately equal to or less that a cross-sectional width of an outer surface of the tubing.

20. The tubing anchor of claim 17, wherein the tubing and the anchor body are bonded together to resist movement of the tubing relative to the anchor body.
